(19) United States
(12) Patent Application Publication
(10) Pub. No.: US 2023/0380663 A1
(43) Pub. Date: Nov. 30, 2023

(54) VENTILATION PIPE SLEEVE SYSTEM, ENDOSCOPE, AND METHOD FOR MANUFACTURING ENDOSCOPE

(71) Applicant: OLYMPUS MEDICAL SYSTEMS CORP., Tokyo (JP)

(72) Inventors: Taihei ONISHI, Yokohama-shi (JP); Hidenosuke HASE, Hachioji-shi (JP); Yumiko AWAU, Setagaya-ku (JP)

(73) Assignee: OLYMPUS MEDICAL SYSTEMS CORP., Tokyo (JP)

(21) Appl. No.: 18/201,808

(22) Filed: May 25, 2023

Related U.S. Application Data
(60) Provisional application No. 63/346,339, filed on May 27, 2022.

(51) Int. Cl. A61B 1/00

(52) U.S. Cl. CPC A61B 1/00057; A61B 1/00103; A61B 1/0011; A61B 1/00137; A61B 1/00119; A61B 1/00128

(57) ABSTRACT

A ventilation pipe sleeve system comprises a ventilation pipe sleeve, a first filter, and a cap attached to the ventilation pipe sleeve. The ventilation pipe sleeve includes a tubular body including an outer surface and an inner surface. The inner surface of the tubular body defines a conduit extending from a first end to a second end of the ventilation pipe sleeve. The first filter is liquid impermeable and is gas permeable, the first filter is fixed to the tubular body to form a watertight structure at a first end region of the tubular body. The cap covers the first filter and at least a portion of the outer surface of the tubular body.

### RELATED APPLICATION DATA

This application is based on and claims priority under 37 U.S.C. § 119 to U.S. Provisional Application No. 63/346,339 filed on May 27, 2022, the entire contents of which are incorporated herein by reference.

### FIELD OF DISCLOSURE

The present disclosure relates to a ventilation pipe sleeve system that is configured to feed gas to an inside of a medical member, an endoscope including the ventilation pipe sleeve system, and a method for manufacturing the endoscope including the ventilation pipe sleeve system.

### BACKGROUND

An endoscope includes an insertion portion to be inserted into a body, such as a stomach, a large intestine, or the like, an operation portion provided on a proximal end side of the insertion portion, and a universal cord extending from a side portion of the operation portion.

An endoscope connecter is provided on a proximal end side of the universal cord.

The endoscope connector is connected to an external device, such as an optical source device or a camera control unit. The endoscope is configured in a watertight manner inside so that immersion in a cleaning liquid or the like to be cleaned and disinfected can be performed after use.

For checking the watertightness, an airtightness inspection of feeding gas to the inside of the endoscope is conducted.

In airtightness testing, gas is fed to the inside of the endoscope via a ventilation pipe sleeve that allows the inside and the outside of the endoscope to communicate.

A conventional ventilation pipe sleeve includes a check valve that delivers air from the inside of the endoscope to the outside.

The check valve is disposed for preventing a flexible tube, such as a bending rubber, which constitutes an outer periphery of a bending portion, from inflating to rupture when the endoscope is left under a negative pressure, for example, when the endoscope is subjected to a sterilization treatment or when the endoscope is transported by air.

Japanese Patent Application Laid-Open Publication No. 2013-46701 (Japanese Patent No. 5659106) discloses a ventilation pipe sleeve including a check valve unit in which a vent valve is pressed in when a cap is attached and removed.

In recent years, a single-use endoscope that is disposed of after being used once has been widely used.

The single-use endoscope is put in a sterilized package after being manufactured and is subjected to a sterilization treatment in a state of being sealed in the sterilized package, and is shipped.

### SUMMARY

A ventilation pipe sleeve system of an embodiment comprises a ventilation pipe sleeve, a first filter, and a cap attached to the ventilation pipe sleeve. The ventilation pipe sleeve includes a tubular body including an outer surface and an inner surface. The inner surface of the tubular body defines a conduit extending from a first end to a second end of the ventilation pipe sleeve. The first filter is liquid impermeable and is gas permeable, the first filter is fixed to the tubular body to form a watertight structure at a first end region of the tubular body. The cap covers the first filter and at least a portion of the outer surface of the tubular body.

A method for manufacturing an endoscope, comprises assembling an endoscope, attaching a ventilation pipe sleeve to a gas feeding connector connected to a gas feeding device, conducting an airtightness inspection, wherein the airtightness inspection includes supplying a gas to an inside of the endoscope via the gas feeding connector and the ventilation pipe sleeve, removing the gas feeding connector from the ventilation pipe sleeve; and attaching a cap to the ventilation pipe sleeve. The endoscope includes an insertion portion, an operation portion disposed on a proximal end side of the insertion portion, a universal cord connected to the operation portion and to an endoscope connector, and a ventilation pipe sleeve system including the ventilation pipe sleeve,

### BRIEF DESCRIPTION OF THE DRAWINGS

FIG. 1 is a perspective view of an endoscope of an embodiment.

FIG. 2 is a partial perspective view of an endoscope connector of a first embodiment.

FIG. 3 is a partial perspective view of the endoscope connector of the first embodiment.

FIG. 4 is a perspective view of a ventilation pipe sleeve of a ventilation pipe sleeve system of the first embodiment.

FIG. 5 is a cross-sectional view of the ventilation pipe sleeve system of the first embodiment.

FIG. 6 is a cross-sectional view of the ventilation pipe sleeve system of the first embodiment.

FIG. 7 is a perspective exploded view of the ventilation pipe sleeve system of the first embodiment.

FIG. 8 is a flowchart of a method for manufacturing an endoscope of the first embodiment.

FIG. 9 is a view for explaining an airtightness inspection of the endoscope of the first embodiment.

FIG. 10A is a perspective view of a cap of a ventilation pipe sleeve system of a modification of the first embodiment.

FIG. 10B is a perspective view of a cap of a ventilation pipe sleeve system of a modification of the first embodiment.

FIG. 11A is a cross-sectional view of a ventilation pipe sleeve system of a modification of the first embodiment.

FIG. 11B is a cross-sectional view of a ventilation pipe sleeve system of a modification of the first embodiment.

FIG. 12 is a perspective exploded view of a ventilation pipe sleeve system of a second embodiment.

FIG. 13 is a cross-sectional exploded view of the ventilation pipe sleeve system of the second embodiment.

### DETAILED DESCRIPTION

### First Embodiment

Hereinafter, embodiments of the present disclosure will be described with reference to the drawings.

Note that the drawings based on the embodiments are schematic views.

The relation between the thickness and width of each portion, the ratio in thickness of each portion, and the like in the drawings may differ from the actual relation, ratio, and the like.

Portions with dimensional relations and ratios differing among the drawings are also included.

Illustration of and assignment of reference signs to some constituent elements are omitted.

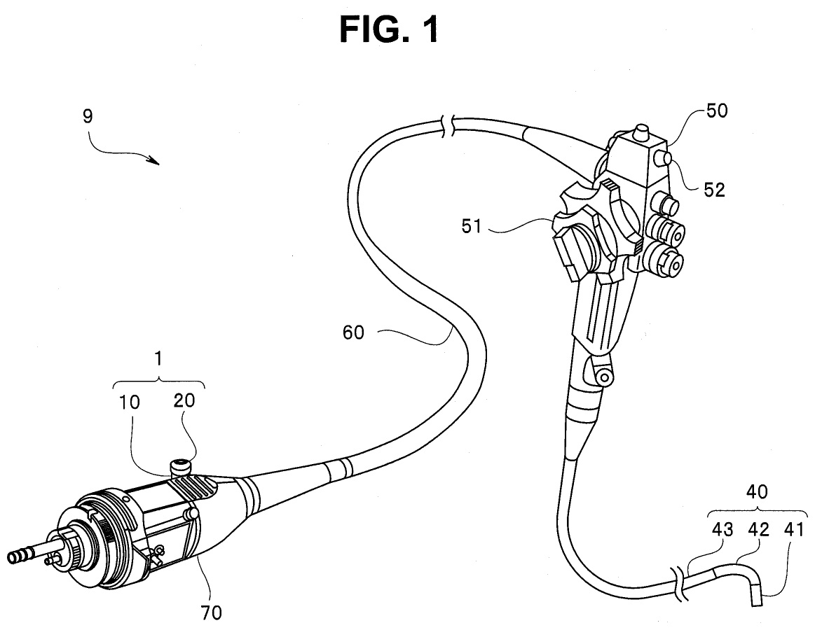

As shown in FIG. 1, an endoscope 9 of the present embodiment includes a ventilation pipe sleeve system 1, an insertion portion 40, an operation portion 50, and a universal cable (universal cord) 60.

The insertion portion 40 is an elongated member to be inserted into a subject.

The insertion portion 40 is configured with a distal end portion 41, a bending portion 42, and a flexible tube portion 43 that are continuously provided.

The operation portion 50 disposed on a proximal end side of the insertion portion 40 is provided with a bending operation portion 51, various switches 52, and the like. The ventilation pipe sleeve 10 is disposed on an outer surface of one of the operation portion 50 and the endoscope connector 70.

An end portion of the universal cable 60 connected to the operation portion 50 is provided with an endoscope connector 70 that is a medical member.

An outer wall of the endoscope connector 70 is provided with a ventilation pipe sleeve 10.

The ventilation pipe sleeve 10 includes a tubular body 11 having a through-hole (conduit) H10 that allows the inside and the outside of the endoscope 9 to communicate.

The ventilation pipe sleeve 10 constitutes the ventilation pipe sleeve system 1 of the present embodiment, together with a cap 20.

In other words, the inside of the endoscope connector 70 and the inside of the operation portion 50 communicate with each other.

The ventilation pipe sleeve 10 may be provided in an outer wall of the operation portion 50 that is a medical member.

In other words, the inside of the endoscope connector 70 and the inside of the operation portion 50 communicate with each other.

The ventilation pipe sleeve 10 may be provided in an outer wall of the operation portion 50 that is a medical member. The ventilation pipe sleeve system 1 comprises the ventilation pipe sleeve 10, the cap 20 and a first filter 30, attached to the ventilation pipe sleeve 10. The ventilation pipe sleeve 10 includes the tubular body 11 including an outer surface and an inner surface. The inner surface of the tubular body 11 defines the conduit H10 extending from a first end to a second end of the ventilation pipe sleeve 10

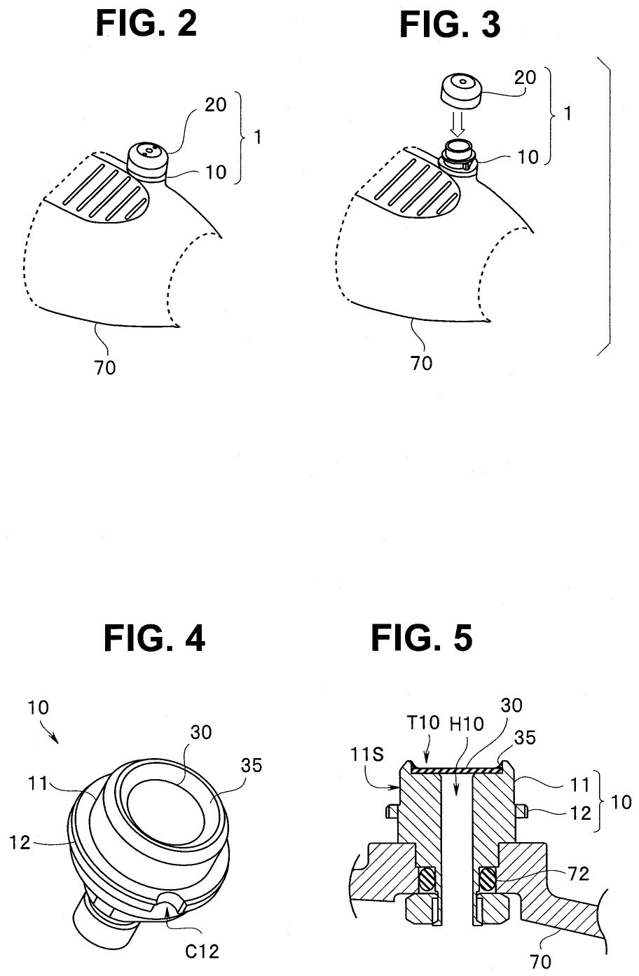

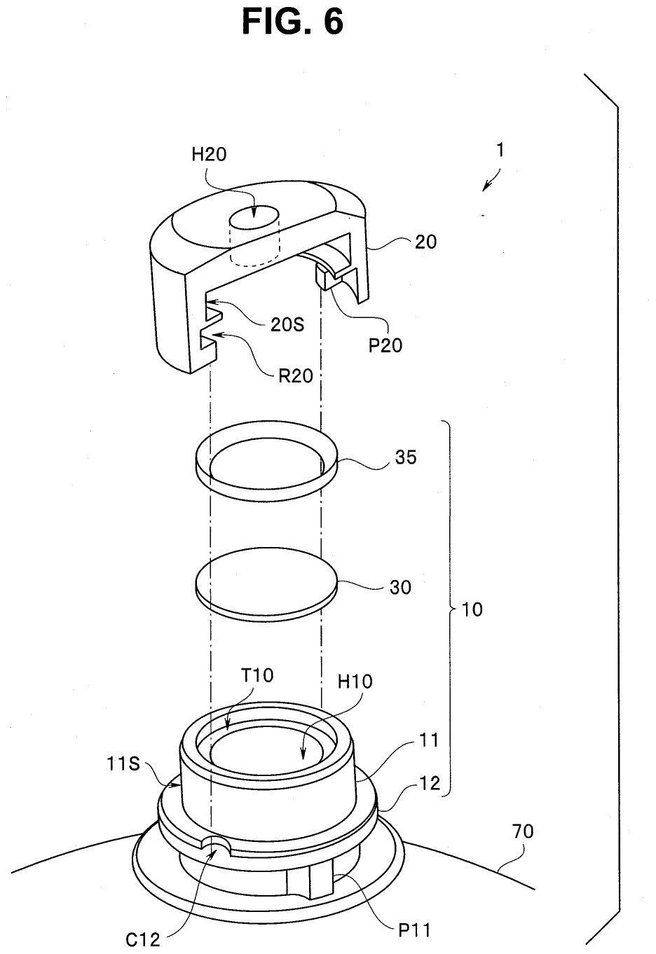

As shown in FIG. 2 to FIG. 7, the cap 20 is fixed to the ventilation pipe sleeve 10.

The cap 20 has a through-hole (hole) H20 that allows the inner surface and the outer surface to communicate.

Thus, although the cap 20 is fixed to the ventilation pipe sleeve 10, ventilation between the inside and the outside is secured. The cap 20 includes a hole H20 providing fluid communication between the inside of the tubular body 11 and the outside of the cap 20.

The cap 20 can be formed of resin, for example, a silicone resin, an ABS resin, a PVC, and rubber.

The ventilation pipe sleeve 10 includes, on an outer surface 11S of the tubular body 11, a protrusion 12 in a ring shape that is an engagement member.

An inner surface of the cap 20 has a recess R20 in a ring shape that engages with the protrusion 12 that is the engagement member of the tubular body 11.

Note that the engagement member of the tubular body 11 may be a recess and the cap 20 may have a protrusion that engages with the engagement member of the tubular body 11.

Further, the recess as the engagement member of the tubular body 11 may be a thread groove and the protrusion of the cap 20 may be a thread that engages with the thread groove. The outer surface of the ventilation pipe sleeve 10 includes a sleeve engagement structure. An inner surface of the cap 20 includes a cap engagement structure, and when the cap 20 is attached to the ventilation pipe sleeve 10, the sleeve engagement structure engages with the cap engagement structure. The sleeve engagement structure includes one or more of a first recess or a first protrusion and the cap engagement structure includes one or more of a second recess or a second protrusion.

The protrusion 12 in a ring shape of the tubular body 11 of the present embodiment is provided with a cutout C12 in two locations.

The inner surface of the cap 20 is provided with a projection P20 in a position corresponding to the cutout C12.

The projection P20 of the cap 20 is configured so as to be inserted into the cutout C12 of the protrusion 12 of the ventilation pipe sleeve 10.

The projection P20 of the cap 20 is set in a position of the cutout C12 of the protrusion 12 of the tubular body 11 and the cap 20 is laid over the tubular body 11, and the cap 20 is then rotated about the center axis of the tubular body 11, so that the projection P20 of the cap 20 abuts on a positioning portion P11 and the cap 20 is fixed to the tubular body 11.

A first filter 30 having liquid impermeability and gas permeability is fixed to an opening on an upper surface of the through-hole H10 of the tubular body 11 in a watertight manner.

The tubular body 11 includes a first cutout T10 that is a ring-shaped groove along the opening on the upper surface of the through-hole H10.

In other words, an end face of the tubular body 11 is provided with a groove having a shape recessed on the center side as compared to a peripheral portion.

An outer periphery of the first filter 30 in a circular shape is housed in the first cutout T10. The outer periphery of the first filter 30 is fixed to the tubular body 11 by means of a watertight first adhesive 35. The first end region of the tubular body 11 includes a first seating surface, and the first filter 30 is located in the first seating surface. The conduit H10 along a main portion has a first inner diameter, a surface of the first seating surface has a second inner diameter. The second inner diameter is larger than the first inner diameter. The first filter 30 is fixed to the tubular body 11 by a first adhesive 35, and the first adhesive 35 is watertight material. The first adhesive is, for example, an epoxy resin or a silicone resin.

The first filter 30 is a porous membrane with a thickness of 0.1 mm having a polyester resin as a base member.

The first filter 30 has a waterproofing property of IPX7 and air permeability of ten seconds in the Gurley number under the JIS L 1096 Gurley test method.

The IPX7 is a protection standard for waterproofing and dustproofing based on the IEC (International Electrotechnical Commission) standard 60529.

The Gurley test method is also specified in the US TAPPI standards and the ISO 5636-5.

The time in seconds taken for a predetermined volume of air (for example, 100 mL) passing through a filter having a predetermined area under a predetermined pressure difference is the Gurley number.

The first filter 30 can have a waterproofing property of IPX7 and 50 seconds or smaller in the Gurley number.

If the first filter 30 is a membrane satisfying the above-described specification, the first filter 30 may be a nonwoven fabric and a net-like film having, as a base member, resin with a large surface tension against water, such as a polytetrafluoroethylene (PTFE) resin. The first filter 30 may also be a stacked film in which a reinforcing film is stacked on a film satisfying the above-described specification.

The inner diameter of the pore of the first filter 30 having a waterproofing property of IPX7 and 50 seconds or smaller in the Gurley number is, for example, 0.1 or smaller.

Therefore, the first filter 30 also has an effect of preventing a foreign object inside the endoscope 9 from being discharged to the outside.

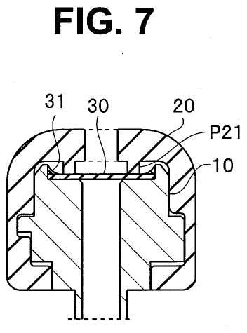

Note that as shown in FIG. 7, the cap 20 can have, on the inner surface, an annular projection (protrusion) P21 that sandwiches and fixes an end portion of the first filter 30 between the annular projection P21 and an outer surface of the ventilation pipe sleeve.

The projection P21 prevents the first filter 30 from deforming outwardly when the air inside the endoscope 9 is exhausted.

If the first filter 30 is fixed to the tubular body 11 in a watertight manner by means of the annular projection P21, the first adhesive 35 is not needed. The inner surface of the cap 20 includes the protrusion P21, and when the cap 20 is attached to the ventilation pipe sleeve 10, the protrusion P21 contacts a surface of the first filter 30.

The cap 20 has a function of preventing the endoscope 9 from being reused as well as protecting the first filter 30.

In other words, as will be described later, the cap 20 is fixed to the ventilation pipe sleeve 10 such that the cap 20 breaks when the cap 20 is removed from the ventilation pipe sleeve 10.

By means of the first filter 30 having a waterproofing property of IPX7, intrusion of water to the inside of the endoscope 9 is prevented.

Further, by means of the first filter 30 having air permeability of 50 seconds or smaller in the Gurley number, damage to a flexible tube or the like inside the endoscope 9 is prevented even when the atmospheric pressure changes during cleaning processing and transportation.

The first filter 30 has a function of a check valve.

Since the ventilation pipe sleeve 10 does not have a complicated mechanism, such as a check valve, the ventilation pipe sleeve 10 is easy to manufacture at a reasonable cost.

Therefore, the ventilation pipe sleeve system 1 can be used for a single-use endoscope that is disposed of after being used once.

While the single-use endoscope enclosed in a sterilized package is transported by air or the like, the ambient pressure decreases.

Therefore, in the endoscope having a ventilation pipe sleeve structured with a check valve, there has been a possibility that a foreign object inside is discharged to the sterilized package.

By contrast, in the endoscope 9 having the ventilation pipe sleeve system 1, a foreign object inside is not discharged to the sterilized package.

<Method for Manufacturing Endoscope>

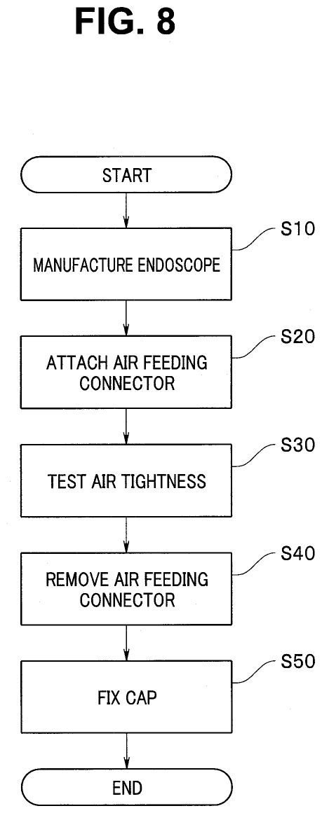

A method for manufacturing the endoscope 9 will be described following a flowchart of FIG. 8.

<Step S10> Manufacturing of Endoscope

The endoscope is manufactured or assembled in which the ventilation pipe sleeve 10 including the tubular body 11 is provided in the operation portion 50 or the endoscope connector 70 of the universal cable 60.

The opening of the through-hole H10 on the upper surface of the tubular body 11 is provided with the outer periphery portion of the first filter 30, which has liquid impermeability and gas permeability, in a watertight manner using the first adhesive 35.

<Step S20> Attachment of Gas Feeding Connector

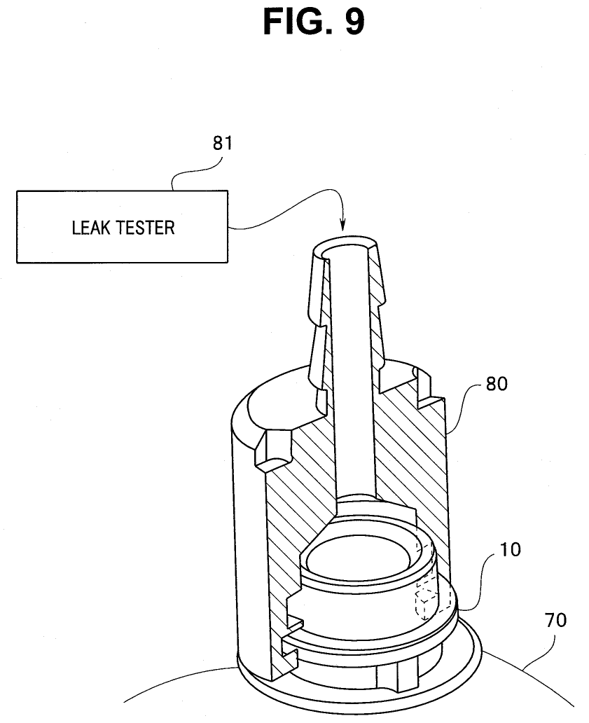

As shown in FIG. 9, for airtightness testing, a gas feeding connector 80 connected to a leak tester (gas feeding device) 81 is attached to the ventilation pipe sleeve 10.

The gas feeding connector 80 includes an inner surface that engages with the engagement member of the ventilation pipe sleeve 10, as with the cap 20.

Therefore, the gas feeding connector 80 is laid over the tubular body 11 and the gas feeding connector 80 is then rotated about the center axis of the tubular body 11, so that the gas feeding connector 80 is fixed to the tubular body 11.

<Step S30> Airtightness Testing

Gas is fed to the inside of the endoscope 9 via the gas feeding connector 80 so that an airtightness inspection is conducted. The airtightness inspection includes supplying a gas to an inside of the endoscope via the gas feeding connector 80 and the ventilation pipe sleeve 10.

For example, the endoscope 9 is immersed in a water tank and securement of the watertightness can be confirmed from, for example, no foam generation of the fed gas.

<Step S40> Removal of Gas Feeding Connector

The gas feeding connector 80 is removed from the ventilation pipe sleeve 10.

<Step S50> Cap Fixation

The cap 20 to protect the first filter 30 is fixed or attached to the ventilation pipe sleeve 10.

The endoscope 9 is a single-use endoscope that is disposed of after being used once.

However, the single-use endoscope could still be reused, against the intention of the manufacturer, by being subjected to the sterilization treatment and the airtightness inspection after use.

For the airtightness inspection of the endoscope, the inspection method or the like is determined on the premise that a predetermined gas feeding device is used.

If the airtightness inspection is conducted using an unexpected gas feeding device, there has been a possibility that the endoscope breaks as well as a possibility that an accurate inspection cannot be conducted.

The cap 20 is fixed to the ventilation pipe sleeve 10 such that when the cap 20 is removed from the ventilation pipe sleeve 10, the cap 20 breaks.

For example, the projection P20 of the cap 20 formed of resin is pushed into the recess of the ventilation pipe sleeve 10 so as to be able to be inserted by snap fitting, but when the cap 20 is attempted to be pulled out, the projection P20 is torn off from the cap 20.

Therefore, the cap 20 cannot be refixed to the ventilation pipe sleeve 10.

Further, if the projection P20 of the cap 20 remains in the ventilation pipe sleeve 10, a new cap to protect the first filter 30 and the gas feeding connector 80 for the airtightness testing cannot be fixed to the ventilation pipe sleeve 10.

The first filter 30 is liquid impermeable and is gas permeable. The first filter 30 is fixed to the tubular body 11 to form a watertight structure at a first end region of the tubular body 11. The cap 20 covers the first filter 30 and at least a portion of the outer surface of the tubular body 11.

The endoscope 9 that is a single-use endoscope is prevented from being reused against the intention of the manufacturer.

### Modifications of the First Embodiment

Ventilation pipe sleeve systems 1A-1D (endoscopes 9A-9D) of modifications of the first embodiment are similar to the ventilation pipe sleeve system 1 (endoscope 9) of the first embodiment and have the same effects.

Therefore, the constituent elements having the same functions are assigned the same reference signs and the descriptions will be omitted.

As shown in FIG. 3, the cap 20 of the ventilation pipe sleeve system 1 has the through-hole H20 on the upper surface.

Therefore, when a foreign object in a needle shape is inserted into the through-hole H20, there has been a possibility that the first filter 30 could break.

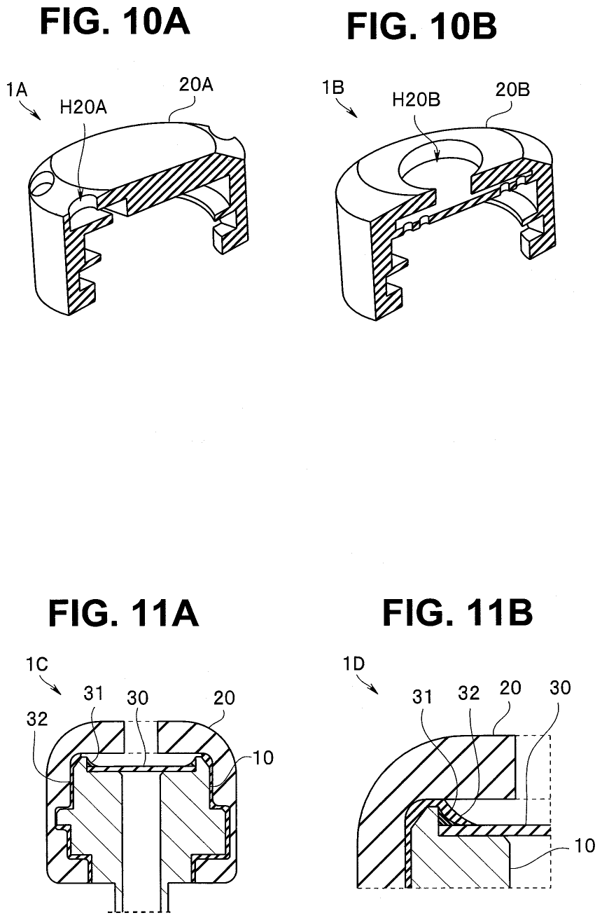

By contrast, a cap 20A of the ventilation pipe sleeve system 1A shown in FIG. 10A has a plurality of insertion holes H20A.

However, the insertion holes H20A each have a step between a hole on the outer surface and a hole on the inner surface.

Thus, when the cap 20A is attached to the ventilation pipe sleeve 10, the first filter 30 cannot be visually recognized from the outside of the cap 20A via the insertion holes H20A.

A cap 20B of the ventilation pipe sleeve system 1B shown in FIG. 10B has one hole H20B on the upper surface.

The cap 20B has, on the inner surface, a plurality of holes that communicate with the hole H20B.

When the cap 20B is attached to the ventilation pipe sleeve 10, the first filter 30 cannot be visually recognized from the outside of the cap 20B via the hole H20B. The first filter 30 is not visible through the hole H20A or H20B from the outside of the cap 20.

In the ventilation pipe sleeve systems 1A and 1B, the first filter 30 cannot be visually recognized from the outside of the cap via the insertion hole.

In the ventilation pipe sleeve systems 1A and 1B, it is less likely that the first filter 30 could break, as compared to the ventilation pipe sleeve system 1.

Note that as long as the configuration is made such that the first filter 30 cannot be visually recognized from the outside via the insertion hole, the insertion hole of the cap may be a through-hole formed in a diagonal orientation or a lateral orientation.

Further, a gap between the cap and the tubular body may have the function of the insertion hole.

In other words, the insertion hole is formed in a size, at an angle, and in a position such that the first filter 30 cannot be contacted from the outside of the cap.

In the ventilation pipe sleeve system 1C shown in FIG. 11A, the cap 20 and the ventilation pipe sleeve 10 are fixed by means of a second adhesive 32.

In other words, the second adhesive 32 is disposed in a gap between the cap 20 and the ventilation pipe sleeve 10. The cap 20 is fixed to the ventilation pipe sleeve 10E by the second adhesive 32.

The second adhesive 32 may be either the same as or different from the first adhesive 35.

The cap 20 fixed by means of the second adhesive 32 is difficult to be removed from the ventilation pipe sleeve 10 without breaking the cap 20.

Therefore, there is no possibility that the endoscope 9C could be reused.

In the ventilation pipe sleeve system 1D shown in FIG. 11B, the second adhesive 32 protrudes to a part of the surface of the first filter 30. The second adhesive 32 can cover the first adhesive 35 and the portion of the surface of the first filter 30.

In the ventilation pipe sleeve system 1D, when the cap 20 is removed from the ventilation pipe sleeve 10, the thin first filter 30 breaks. Thus, the above-described airtightness testing cannot be conducted again on the ventilation pipe sleeve system 1D. Accordingly, there is no possibility that the endoscope 9D could be reused.

### Second Embodiment

A ventilation pipe sleeve system 1E (endoscope 9E) of a second embodiment is similar to the ventilation pipe sleeve system 1 (endoscope 9) of the first embodiment and has the same effects.

Therefore, the constituent elements having the same functions are assigned the same reference signs and the descriptions will be omitted.

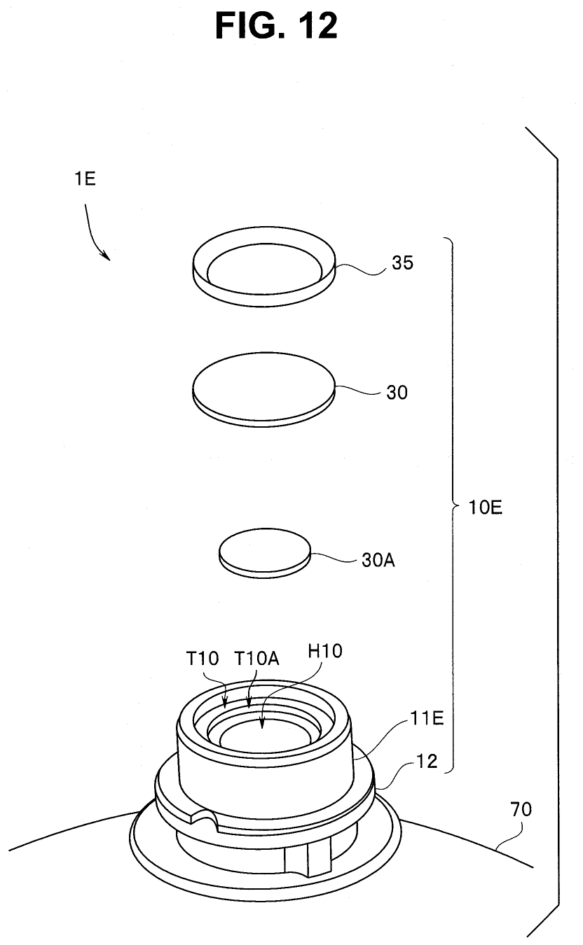

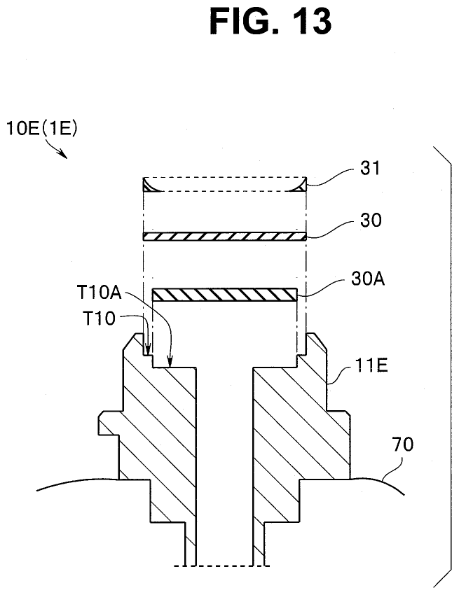

As shown in FIG. 12 and FIG. 13, a tubular body 11E of the ventilation pipe sleeve system 1E includes, along the first cutout T10, a second cutout T10A that is a ring-shaped groove smaller than the first cutout T10.

A ventilation pipe sleeve 10E further includes a second filter 30A with the outer periphery housed in and fixed to the second cutout T10A and having gas permeability.

The material of the second filter 30A may be either the same as or different from the material of the first filter 30.

For example, the second filter 30A may be a reinforcing member thicker than the first filter 30 and having a plurality of large pores, which reinforces the thin first filter if the second filter 30A is excellent in gas permeability.

The ventilation pipe sleeve system 1E including the second filter 30A having the same liquid impermeability and gas permeability as the liquid impermeability and the gas permeability of the first filter 30 is more reliable than the ventilation pipe sleeve system 1.

Meanwhile, in the ventilation pipe sleeve system 1E including the second filter that is a reinforcing member, the first filter 30 is prevented from breaking. The first end region of the conduit H10 includes a second recess T10A, the second filter 30A can be located in a second seating surface of the tubular body 11, and the second filter is gas permeable. A surface of the second seating surface has a third inner diameter. The third inner diameter can be larger than the first inner diameter of the conduit H10 and the third inner diameter can be smaller than the second inner diameter of the first seating surface. The first seating surface can be closer to an end surface of the first end region of the conduit than the second seating surface.

The endoscope 9 described in the above embodiments is a single-use endoscope that is disposed of after being used once, but the endoscope of the present disclosure may be a reusable endoscope that is repeatedly used.

Although the endoscope 9 is a flexible endoscope in which the insertion portion 40 has flexibility, the endoscope of the present disclosure may be a rigid endoscope having a rigid straight tube as the insertion portion.

The endoscope 9 is not limited to medical use but may be for industrial use.

The present disclosure is not limited to the embodiment explained above. Various changes, alterations, and the like are possible within a range not changing the gist of the disclosure.
Example 1. A ventilation pipe sleeve system comprising:
   a ventilation pipe sleeve disposed on an outer wall of a medical member, the ventilation pipe sleeve including a tubular body including a through-hole that allows an inside and an outside of the medical member to communicate;
   a first filter having liquid impermeability and gas permeability, the first filter being fixed to an opening of the tubular body in a watertight manner; and
   a cap that protects the first filter, the cap being fixed to the tubular body.
Example 2. The ventilation pipe sleeve system according to Example 1,
   wherein
   the ventilation pipe sleeve comprises an engagement member on an outer surface of the tubular body, and
   an inner surface of the cap engages with the engagement member.
Example 3. The ventilation pipe sleeve system according to Example 2,
   wherein
   the engagement member is a recess or a protrusion provided in the ventilation pipe sleeve, and
   the inner surface of the cap is in a shape that engages with the recess or the protrusion.
Example 4. The ventilation pipe sleeve system according to Example 1, wherein an inner surface of the cap is provided with a projection that sandwiches and fixes the first filter between the projection and an outer surface of the ventilation pipe sleeve.
Example 5. The ventilation pipe sleeve system according to Example 1,
   wherein
   the tubular body comprises a first cutout in a ring shape along the opening, and
   an outer periphery of the first filter in a circular shape is housed in the first cutout.
Example 6. The ventilation pipe sleeve system according to Example 5, wherein the first filter is fixed to the tubular body by means of a watertight first adhesive.
Example 7. The ventilation pipe sleeve system according to Example 1, wherein the cap and the ventilation pipe sleeve are fixed by means of a second adhesive.
Example 8. The ventilation pipe sleeve system according to Example 7, wherein the second adhesive protrudes to a part of a surface of the first filter.
Example 9. The ventilation pipe sleeve system according to Example 5,
   wherein
   the tubular body comprises a second cutout in a ring shape along the first cutout, the second cutout being smaller than the first cutout, and
   a second filter having gas permeability is further included, and an outer periphery of the second filter is housed in the second cutout.
Example 10. The ventilation pipe sleeve system according to Example 1, wherein the cap comprises one or more insertion holes that allow an inner surface and an outer surface to communicate.
Example 11. The ventilation pipe sleeve system according to Example 10, wherein the first filter is provided in a position where the first filter cannot be visually recognized from an outside of the cap via the insertion hole.
Example 12. The ventilation pipe sleeve system according to Example 1, wherein the medical member is an operation portion of an endoscope or an endoscope connector of a universal cord.
Example 13. The ventilation pipe sleeve system according to Example 1, wherein the first filter has a waterproofing property of IPX7 and air permeability of 50 seconds or smaller in a Gurley number.
Example 14. An endoscope comprising:
   an insertion portion inserted into a subject;
   an operation portion disposed on a proximal end side of the insertion portion;
   a universal cord connected to the operation portion and including an endoscope connector; and
   a ventilation pipe sleeve system disposed in the operation portion or the endoscope connector,
   wherein
   the ventilation pipe sleeve system comprises:
   a ventilation pipe sleeve disposed on an outer wall of a medical member, the ventilation pipe sleeve including a tubular body including a through-hole that allows an inside and an outside of the medical member to communicate;
   a first filter having liquid impermeability and gas permeability, the first filter being fixed to an opening of the tubular body in a watertight manner; and
   a cap that protects the first filter, the cap being fixed to the ventilation pipe sleeve.
Example 15. The endoscope according to Example 14, wherein the endoscope is a single-use endoscope that is disposed of after being used once.
Example 16. A method for manufacturing an endoscope, comprising:
   manufacturing an endoscope in which a ventilation pipe sleeve including a tubular body is provided in an operation portion or an endoscope connector of a universal cord, wherein a first filter is fixed to an opening of a through-hole of the tubular body in a watertight manner, the first filter having liquid impermeability and gas permeability;
   attaching, to the ventilation pipe sleeve, a gas feeding connector connected to a gas feeding device;
   conducting an airtightness inspection such that gas is fed to an inside of the endoscope via the gas feeding connector;
   removing the gas feeding connector from the ventilation pipe sleeve; and
   fixing a cap to the ventilation pipe sleeve.
Example 17. The method for manufacturing an endoscope according to Example 16, wherein the first filter is fixed to the tubular body by means of a watertight first adhesive.
Example 18. The method for manufacturing an endoscope according to Example 16,
   wherein
   the ventilation pipe sleeve comprises an engagement member on an outer surface of the tubular body, and
   an inner surface of the cap that engages with the engagement member rotates about a center axis of the tubular body so that the cap is fixed to the ventilation pipe sleeve.
Example 19. The method for manufacturing an endoscope according to Example 16,
   wherein
   the endoscope is a single-use endoscope that is disposed of after being used once, and
   when the cap is removed from the ventilation pipe sleeve, at least one of the cap or the first filter breaks.
Example 20. The method for manufacturing an endoscope according to Example 16, wherein the cap is fixed to the ventilation pipe sleeve by means of a second adhesive.
Example 21. A method for manufacturing an endoscope, comprising:
   assembling an endoscope, wherein the endoscope includes an insertion portion, an operation portion disposed on a proximal end side of the insertion portion, a universal cord connected to the operation portion and to an endoscope connector, and a ventilation pipe sleeve system including a ventilation pipe sleeve;
   attaching the ventilation pipe sleeve to a gas feeding connector connected to a gas feeding device;
   conducting an airtightness inspection, wherein the airtightness inspection includes supplying a gas to an inside of the endoscope via the gas feeding connector and the ventilation pipe sleeve;
   removing the gas feeding connector from the ventilation pipe sleeve; and
   attaching a cap to the ventilation pipe sleeve.
Example 22. The method according to Example 21, wherein the ventilation pipe sleeve system includes:
   the ventilation pipe sleeve, and
   a first filter,
   wherein the ventilation pipe sleeve includes a tubular body including an outer surface and an inner surface,
   wherein the inner surface of the tubular body defines a conduit extending from a first end to a second end of the ventilation pipe sleeve,
   wherein the first filter is liquid impermeable and is gas permeable,
   wherein the first filter is fixed to the tubular body to form a watertight structure at a first end region of the tubular body, and
   wherein, when the cap is attached to the ventilation pipe sleeve, the cap covers the first filter and at least a portion of the outer surface of the tubular body.
Example 23. The method according to Example 21, wherein the ventilation pipe sleeve is on an outer surface of one of the operation portion and the endoscope connector.
Example 24. The method according to Example 22, wherein the first filter is fixed to the tubular body by a first adhesive, and wherein the first adhesive is watertight.
Example 25. The method according to Example 22, wherein the outer surface of the ventilation pipe sleeve includes a sleeve engagement structure,
   wherein an inner surface of the cap includes a cap engagement structure, and
   wherein attaching the cap to the ventilation pipe sleeve includes rotating the cap relative to the tubular body to engage the sleeve engagement structure with the cap engagement structure.
Example 26. The method according to Example 25, wherein the sleeve engagement structure includes one or more of a first recess or a first protrusion and the cap engagement structure includes one or more of a second recess or a second protrusion.
Example 27. The method according to Example 22, wherein the endoscope is a single-use endoscope,
   wherein the method further includes removing the cap from the ventilation pipe sleeve, and
   wherein removing the cap from the ventilation pipe sleeve breaks one or more of the cap or the first filter.
Example 28. The method for manufacturing an endoscope according to Example 24, wherein the cap is fixed to the ventilation pipe sleeve by a second adhesive.

What is claimed is:

1. A ventilation pipe sleeve system, comprising:
a ventilation pipe sleeve;
a first filter; and
a cap attached to the ventilation pipe sleeve,
wherein the ventilation pipe sleeve includes a tubular body including an outer surface and an inner surface,
wherein the inner surface of the tubular body defines a conduit extending from a first end to a second end of the ventilation pipe sleeve,
wherein the first filter is liquid impermeable and is gas permeable,
wherein the first filter is fixed to the tubular body to form a watertight structure at a first end region of the tubular body, and
wherein the cap covers the first filter and at least a portion of the outer surface of the tubular body.

2. The ventilation pipe sleeve system according to claim 1, wherein the outer surface of the tubular body includes a sleeve engagement structure,
wherein an inner surface of the cap includes a cap engagement structure, and
wherein, when the cap is attached to the ventilation pipe sleeve, the sleeve engagement structure engages with the cap engagement structure.

3. The ventilation pipe sleeve system according to claim 2, wherein the sleeve engagement structure includes one or more of a first recess or a first protrusion and the cap engagement structure includes one or more of a second recess or a second protrusion.

4. The ventilation pipe sleeve system according to claim 1, wherein an inner surface of the cap includes a protrusion, and
wherein, when the cap is attached to the ventilation pipe sleeve, the protrusion contacts a surface of the first filter.

5. The ventilation pipe sleeve system according to claim 1, wherein the first end region of the tubular body includes a first seating surface, and
wherein the first filter is located in the first seating surface.

6. The ventilation pipe sleeve system according to claim 5, wherein the conduit along a main portion has a first inner diameter,
wherein a surface of the first seating surface has a second inner diameter, and
wherein the second inner diameter is larger than the first inner diameter.

7. The ventilation pipe sleeve system according to claim 1, wherein the first filter is fixed to the tubular body by a first adhesive, and wherein the first adhesive is watertight material.

8. The ventilation pipe sleeve system according to claim 7, wherein the cap is fixed to the ventilation pipe sleeve by a second adhesive.

9. The ventilation pipe sleeve system according to claim 8, wherein the second adhesive covers the first adhesive and a portion of a surface of the first filter.

10. The ventilation pipe sleeve system according to claim 6, wherein the first end region of the tubular body includes a first recess,
wherein a second filter is located in a second seating surface of the tubular body, and
wherein the second filter is gas permeable.

11. The ventilation pipe sleeve system according to claim 10, wherein a surface of the second seating surface has a third inner diameter, and
wherein the third inner diameter is larger than the first inner diameter and the third inner diameter is smaller than the second inner diameter.

12. The ventilation pipe sleeve system according to claim 11, wherein the first seating surface is closer to an end surface of the first end region of the tubular body than the second seating surface.

13. The ventilation pipe sleeve system according to claim 1, wherein the cap includes a hole providing fluid communication between an inside of the tubular body and an outside of the cap.

14. The ventilation pipe sleeve system according to claim 13, wherein the first filter is not visible through the hole from the outside of the cap.

15. The ventilation pipe sleeve system according to claim 1, wherein the first filter has a waterproofing property of IPX7 and an air permeability of 50 seconds or smaller in a Gurley number.

16. An endoscope, comprising:
an insertion portion;
an operation portion disposed proximally relative to the insertion portion;
a universal cord connected to the operation portion and to an endoscope connector; and
a ventilation pipe sleeve system according to claim 1,
wherein the ventilation pipe sleeve is disposed on an outer surface of one of the operation portion and the endoscope connector.

17. The endoscope according to claim 16, wherein the endoscope is a single-use endoscope.

18. A method for manufacturing an endoscope, comprising:
assembling an endoscope, wherein the endoscope includes an insertion portion, an operation portion disposed proximally relative to the insertion portion, a universal cord connected to the operation portion and to an endoscope connector, and a ventilation pipe sleeve system including a ventilation pipe sleeve;
attaching the ventilation pipe sleeve to a gas feeding connector connected to a gas feeding device;
conducting an airtightness inspection, wherein the airtightness inspection includes supplying a gas to an inside of the endoscope via the gas feeding connector and the ventilation pipe sleeve;
removing the gas feeding connector from the ventilation pipe sleeve; and
attaching a cap to the ventilation pipe sleeve.

19. The method according to claim 18, wherein the ventilation pipe sleeve system includes:
the ventilation pipe sleeve, and
a first filter,
wherein the ventilation pipe sleeve includes a tubular body including an outer surface and an inner surface,
wherein the inner surface of the tubular body defines a conduit extending from a first end to a second end of the ventilation pipe sleeve,
wherein the first filter is liquid impermeable and is gas permeable,
wherein the first filter is fixed to the tubular body to form a watertight structure at a first end region of the tubular body, and
wherein, when the cap is attached to the ventilation pipe sleeve, the cap covers the first filter and at least a portion of the outer surface of the tubular body.

20. The method according to claim 18, wherein the ventilation pipe sleeve is on an outer surface of one of the operation portion and the endoscope connector.
